(19) United States
(12) Patent Application Publication
(10) Pub. No.: US 2026/0348986 A1
(43) Pub. Date: Nov. 5, 2026

(54) HORMONE TESTING

(71) Applicant: WLNESS SCIENCE LTD, Weybridge Surrey (GB)

(72) Inventors: Jasmine TAGESSON, Weybridge Surrey (GB); Karolina LÖFQVIST, Weybridge Surrey (GB); Richard CAMBELL, Weybridge Surrey (GB); Rhea PEREZ, Weybridge Surrey (GB)

(21) Appl. No.: 19/120,349

(22) PCT Filed: Oct. 13, 2023
(86) PCT No.: PCT/GB2023/052667

§ 371 Date: Apr. 11, 2025

Related U.S. Application Data
(60) Provisional application No. 63/379,532, filed on Oct. 14, 2022.

(51) Int. Cl. G01N 33/74; G01N 33/543; G01N 33/76

(52) U.S. Cl. CPC G01N 33/743; G01N 33/54388; G01N 33/76; G01N 2333/575; G01N 2333/59

(57) ABSTRACT

A system and method for determining a hormone profile of a user is disclosed herein, the system comprising: a hormone test set; and a monitor, wherein: the hormone test set comprises a plurality of hormone tests and each of the plurality of hormone tests is configured to be used to receive a sample from a user, detect a hormone in the sample, and display a test result indicative of the concentration of the hormone in the sample; the monitor is able to capture an image of the test result, determine, quantitatively, the concentration of the hormone based on the image of the test result, store the determined concentration in a memory, and produce an output based on the determined concentration; and the monitor is further configured to receive an input from the user and, based on the input, determine and notify the user as to which of the plurality of hormone tests to provide a sample to and capture an image of on which day of the user's current menstrual cycle.

The present invention relates to a system and method for determining a hormone profile of a user, in particular to a system and method for determining a hormone profile of a user which can be used at home.

Various tests exist to measure the hormone levels of a user. Some tests may be qualitative, outputting an indication of hormone level, for example a “high” or “low” output based on a threshold. An example of such a test is a pregnancy test that indicates a positive output based upon levels of human chorionic gonadotrophin (hCG) above a detection threshold. Some tests may be quantitative, providing an output indicative of an actual hormone level in the sample. Different hormone tests can be based on different bodily fluids, including blood, saliva and/or urine. Quantitative tests are often carried out by a doctor.

It is known to monitor certain hormone test results using a system to allow a user to detect fertile days, ovulation, determine ‘safe’ days for contraception or to monitor fertility.

The present invention provides a system for determining a hormone profile of a user, the system comprising:
a hormone test set; and
a monitor, wherein:
the hormone test set comprises a plurality of hormone tests and each of the plurality of hormone tests is configured to be used to receive a sample from a user, detect a hormone in the sample, and display a test result indicative of the concentration of the hormone in the sample;
the monitor is able to capture an image of the test result, determine, quantitatively, the concentration of the hormone based on the image of the test result, store the determined concentration in a memory, and produce an output based on the determined concentration; and
the monitor is further configured to receive an input from the user and, based on the input, determine and notify the user as to which of the plurality of hormone tests to use and capture an image of on which day of the user's current menstrual cycle.

This provides a personalised hormone testing system which, dependent on the individual user, determines on which days of the user's current menstrual cycle a hormone test should be conducted and for how many days during a user's cycle a hormone test should be conducted. As discussed below, the set of hormone tests may comprise hormone tests for a plurality of hormones and the monitor may determine on which days of the user's menstrual cycle a hormone test for a particular hormone should be conducted and for how many days during a user's cycle a hormone test for a particular hormone should be conducted. The monitor may determine a schedule for one or more hormone tests to detect one of more hormones for the user's current menstrual cycle and/or one or more menstrual cycles. The determined testing schedule may cover at least one menstrual cycle, or at least three menstrual cycles.

The test is quantitative meaning that the monitor can analyse the image of the test result to determine a hormone level in the sample, for example the concentration of hormone in the sample, at least for concentrations within a predetermined range of concentrations. It should be understood that the concentration of hormone in the sample may be the concentration of the hormone itself, or a concentration of a metabolite of a hormone in the sample. The concentration of hormone in the sample, or the concentration of a metabolite of a hormone in the sample, is directly related to the level of the hormone in the body of the user. The output may comprise the hormone level in the sample and/or the hormone level in the body of the user, for example the concentration of hormone in the blood of the user. The determined quantitative result value may have an error of less than 20%, less than 15%, less than 10%, less than 7.5%, less than 5%, less than 2.5%, or less than 1.5% when compared with the true value. The error in the quantitative result value may be above or below the true value. This error value may be the same for each test, or may vary between tests or test types.

The results are stored in a memory, which may be a solid state memory or hard drive within the device, or the memory may be a remote storage, for example storage on a user's computer or cloud storage to which the monitor can transmit the results. The transmission may be wired, wireless, or a combination of these, or other methods.

As the results are stored in memory, past results can be recalled and presented against time to produce a hormone profile for that user over time. The test results may be supplemented by predicted results for days on which a test was not, or has not yet been, carried out based on the anticipated hormone variations during a user's menstrual cycle. Health data such as data collected by third party devices, such as pulse meters or blood pressure monitors, or apps, such as Apple Health™ and Sleep Cycle™, may be integrated with the results to provide further information as to the health of the user.

The hormone test set comprises a plurality of hormone tests and each of said hormone test may test for one or more hormones simultaneously. Each hormone test may to test for a single hormone. This can be cost effective as this allows the monitor to prompt a user to test only one specific hormone level on a particular day and the user can select the appropriate single test so unnecessary tests are not carried out.

The test result may be any indication produced by the hormone test an image of which can be analysed to determine a quantitative hormone level. The result may comprise a colour change, with the final colour being dependent upon the hormone level in the sample. The result may comprise a test region within which an intensity of final colour is dependent upon the hormone level in the sample.

The monitor is able to capture an image of a test result and the monitor may comprise a camera or other imaging device so that the monitor can capture an image directly. The monitor may capture an image indirectly, for example it may be connected to a camera or other imaging device, or source of images captured by such a device. The monitor may be connected in a wired or wireless manner.

The monitor is able to determine, quantitatively, the concentration of the hormone in the sample based on the image of the test result. This determination may be based on an intensity, size, colour or other feature of a revealed test mark. This determination may be based on a ratio of a feature of a test mark and a feature of a control mark.

The sample may be any suitable biological sample which can be tested to determine a hormone level, for example blood, saliva or urine. The sample may be urine as this provides a convenient source of sample material to test. A urine sample may be collected mid-flow during urination, or may be a urine sample that is collected, for example in a cup, and then applied to a test region of a hormone test to carry out the test.

The image of the test result may be an image in visible light, ultra violet or infra-red, or a combination of these in which the test result can be detected. Capturing different wavelengths may facilitate the quantitative analysis of the test results in the image by the monitor.

The output may comprise a numerical value for the level, for example concentration, of the tested hormone, a graph or table over time comprising the test result together with previous test results retrieved from memory and/or predicted or calculated results, predictions of future results or other data such as symptoms that might be experienced, and/or a graphical representation of the hormone level, for example a low/medium/high indication.

The input from the user may comprise any day/date of the user's menstrual cycle that would be recognised by the user. The input may include one or more of the date of the first day of the current menstrual cycle; the date of the first day of a preceding menstrual cycle, for example the menstrual cycle immediately preceding the current cycle or any preceding menstrual cycle; a duration of the user's cycle, whether the user's cycle is regular or not. The input may also comprise symptoms experienced, for example, physical symptoms such as discomfort, pain or headaches, or mental symptoms such as stress, anxiety, low mood or brain fog, and on what date they were experienced.

The monitor, based on the input from a user, determines and notifies the user on which days of the user's current menstrual cycle the user should use one or more of the plurality of hormone tests and then use the monitor to capture an image of the test result. The notification may comprise one or more of a schedule to which the user can refer, a daily alert, alarm or other indication to inform the user. The monitor may also provide prompts and/or reminders. The monitor may provide a window of one or more days within which a test for a particular hormone should be carried out. In some cases the window may be a specified day of the user's current menstrual cycle. The determination of the days on which to use which test based on the input from a user may be based on a look-up table, database or algorithm. A day determined based on an initial input may be varied based on further input from a user.

As set out above, the input may comprise the first date of the user's current menstrual cycle. The input from the user may further comprise the first date of the user's previous menstrual cycle. This allows the monitor to calculate the duration, in number of days, of the user's cycle. The monitor may be configured to determine and notify the user as to which of the plurality of hormone tests to use and capture an image of the test result on which day based on the number of days of the user's previous menstrual cycle. The duration of the users previous menstrual cycle may provide an indication of the likely duration and timing of the current menstrual cycle, particularly if the user have regular menstrual cycles.

The plurality of hormone tests may comprise a first hormone test set comprising at least one first hormone test adapted to test a level of a first hormone. The first hormone test set may be adapted to test a level of a first hormone. The first hormone test set may comprise all of the plurality of hormone tests of the hormone test set or may comprise a subset of plurality of hormone tests of the hormone test set.

The first hormone test set may comprise a plurality of first hormone tests. The user may not be notified to use one of the plurality of first hormone tests and capture an image of the test result on every day of the user's current menstrual cycle. In this way the monitor can notify the user to test a particular hormone only when necessary, for example in order to produce or monitor a hormone profile or monitor a change, without using tests when not required. This reduces waste and may be more efficient for the user as testing on every day may not be required.

In addition to the first hormone test set, the plurality of hormone tests may further comprise a second hormone test set comprising at least one second hormone test adapted to test a level of a second hormone. The first hormone and second hormone are different hormones.

The second hormone test set may comprise a plurality of second hormone tests. The user may not be notified to use and capture an image of one of the plurality of second hormone tests on every day of the user's current menstrual cycle.

The plurality of hormone tests may further comprise a third hormone test set comprising at least one third hormone test adapted test a third hormone level, wherein the first hormone, second hormone and third hormone are different hormones.

The hormones being measured by the hormone tests of the system may include follicle-stimulating hormone, estrogen, progesterone, luteinising hormone, testosterone, Anti-Müllerian Hormone (AMH), thyroid-stimulating hormone (TSH), triiodothyronine (T3), thyroxine (T4) and cortisol. It should be understood that the hormone test set may comprise tests for one, some or all of these hormones. The set of hormone tests may comprise hormone tests for follicle-stimulating hormone (FSH), estrogen and progesterone.

The first hormone may be follicle-stimulating hormone (FSH), the second hormone may be estrogen, measured for example via estrone-3-glucuronide (E3G), and the third hormone may be progesterone, measured for example via pregnanediol glucuronide (PDG). Monitoring the levels of these three hormones during a user's menstrual cycle, particularly over more than one cycle, for example three cycles, provides a good indication of a user's hormonal health. The system may permit a user to monitor their hormones as described for a period of at least a year, for at least 2 years, or for 3 or more years.

The monitor may be configured to notify the user to use and capture an image of the test result of the first hormone test within a window, for example a day, when the user's level of follicle-stimulating hormone is expected to start increasing. Detecting a sufficient level of follicle-stimulating hormone during this window indicates that the ovaries will soon start producing estrogen.

The monitor may be configured to notify the user to use and capture an image of the test result of the second hormone test on days when the user's level of estrogen (measured for example via estrone-3-glucuronide) is expected to peak. Detecting a sufficient level of estrogen during the first window ensures estrogen production was triggered and that ovulation is likely to occur soon.

The monitor may be configured to notify the user to use and capture an image of the test result of the third hormone test on the day when the user's level of progesterone (measured for example via pregnanediol glucuronide PDG) is expected to rapidly increase. Detecting a sufficient level of progesterone during this window indicates that ovulation took place. Testing the user's levels of estrogen and progesterone within the same window is beneficial as the ratio between these hormones may be connected to symptoms of premenstrual syndrome, anovulation, PCOS and/or other conditions.

The days on which the user is notified to test may be dependent on the total number of days of the user's previous menstrual cycle.

To be able to determine that hormones are within a normal range indicating a good hormonal balance, for a first set of users a minimum test schedule below may be used.

The first set of users have the following characteristics. They have no underlying medical condition and they have a regular menstrual cycle within a range 15-60 days, (allowable variation 7-9 days depending upon age-aged 18-25 y≤9 d; 26-41 y, ≤7 d; and for 42-45 y, ≤9 d).

The minimum test schedule for the first set of users may comprise the following hormone tests:
FSH—1 test per cycle
E3G—2 tests per cycle
PDG—1 test per cycle

The test schedule, based on days from the start of the user's menstrual cycle, wherein the start of the user's menstrual cycle is the first day of a period, may be as follows:
“Normal” cycles—cycle length between 24 to 38 days

| | Av Cycle length | | | | | | | | | | | | | | |
|---|---|---|---|---|---|---|---|---|---|---|---|---|---|---|---|
| | 24 | 25 | 26 | 27 | 28 | 29 | 30 | 31 | 32 | 33 | 34 | 35 | 36 | 37 | 38 |
| FSH | 2 | 2 | 2 | 3 | 3 | 3 | 3 | 3 | 3 | 3 | 3 | 3 | 3 | 3 | 3 |
| E3G | 11 | 12 | 13 | 14 | 14 | 15 | 15 | 16 | 17 | 18 | 18 | 19 | 20 | 21 | 22 |
| E3G | 18 | 19 | 20 | 21 | 21 | 22 | 22 | 23 | 24 | 25 | 25 | 26 | 27 | 28 | 29 |
| PDG | 18 | 19 | 20 | 21 | 21 | 22 | 22 | 23 | 24 | 25 | 25 | 26 | 27 | 28 | 29 |

“Frequent” cycles—cycle length between 15 and 23 days

| | Av Cycle length | | | | | | | | |
|---|---|---|---|---|---|---|---|---|---|
| | 15 | 16 | 17 | 18 | 19 | 20 | 21 | 22 | 23 |
| FSH | 2 | 2 | 2 | 2 | 2 | 2 | 2 | 2 | 2 |
| E3G | 6 | 6 | 7 | 7 | 8 | 8 | 9 | 9 | 10 |
| E3G | 12 | 13 | 14 | 15 | 16 | 16 | 17 | 18 | 18 |
| PDG | 12 | 13 | 14 | 15 | 16 | 16 | 17 | 18 | 18 |

“Infrequent” cycles—cycle length between 39 to 60 days

| | Av Cycle length | | | | | | | | | |
|---|---|---|---|---|---|---|---|---|---|---|
| | 39 | 40 | 41 | 42 | 43 | 44 | 45 | 46 | 47 | 48 |
| FSH | 3 | 3 | 3 | 3 | 3 | 3 | 3 | 3 | 3 | 3 |
| E3G | 17 | 18 | 19 | 20 | 21 | 22 | 23 | 24 | 25 | 26 |
| E3G | 24 | 25 | 26 | 27 | 28 | 29 | 30 | 31 | 32 | 33 |
| PDG | 24 | 25 | 26 | 27 | 28 | 29 | 30 | 31 | 32 | 33 |

| | Av Cycle length | | | | | | | | | | | |
|---|---|---|---|---|---|---|---|---|---|---|---|---|
| | 49 | 50 | 51 | 52 | 53 | 54 | 55 | 56 | 57 | 58 | 59 | 60 |
| FSH | 3 | 3 | 3 | 3 | 3 | 3 | 3 | 3 | 3 | 3 | 3 | 3 |
| E3G | 27 | 28 | 29 | 30 | 31 | 32 | 33 | 34 | 35 | 36 | 37 | 38 |
| E3G | 34 | 35 | 36 | 37 | 38 | 39 | 40 | 42 | 42 | 43 | 44 | 45 |
| PDG | 34 | 35 | 36 | 37 | 38 | 39 | 40 | 41 | 42 | 43 | 44 | 45 |

The days above may be the day on which a user is notified to test the specified hormone, or may be used as a start, end or midpoint of a testing window which may comprise a plurality of days.

A second set of users may comprise those users with an underlying medical condition, or those with irregular menstrual cycles, and the monitor may use tables, algorithms, or a combination of these to determine a hormone testing schedule based on initial user input.

The hormone testing schedule may be modified based upon subsequent user input or received test results.

A user may be notified, or instructed, to capture a sample early in their day. For a urine test the user may be notified, or instructed, to take the sample of the first urination of the day.

Each of the plurality of hormone tests may be a lateral flow test. Each of the lateral flow tests, in response to receiving a sample to test, may display a control mark and a test mark, for example, a control line and a test line. The test result may comprise the test mark, or test line, and the colour intensity of the test mark or test line may be indicative of the level, for example, the concentration, of the hormone in the sample. The control mark and test mark may appear on a background. The background and colours of the control mark and test mark may be selected to enhance the contrast between the background and the test and control lines. The background may be white and the test and control marks may be red, green or blue. The lateral flow test may comprise a test region adapted to collect a mid-flow sample of urine from a user as the user urinates or a sample of urine which has previously been collected in a cup. The test region may be at an end of the test. The test may provide a quantitative result within a particular range and may provide a qualitative result outside of that range. For example, a quantitative result may be provided within the range of 0-140 mIU/mL for FSH, 0-500 ng/mL for E3G and/or 0-50 ug/mL for PDG (for example, by producing a test line with a particular intensity), and a qualitative result over 140 mIU/mL for FSH, 500 ng/mL for E3G and/or 50 ug/mL for PDG (for example, by not producing a test line, or a test line with an intensity below a given threshold.

The lateral flow test may comprise an orientation marker. The orientation marker may be formed into a housing and/or a cap of the test, printed thereon, and/or comprise the control or test mark. The control and test marks may appear in a result region of the test and the orientation marker may be in or adjacent the result region. The monitor may identify the test mark by identifying the orientation marker in the image of the test result and using that to determine a location of the test mark. The monitor may be able to identify the location of the test mark directly based on the orientation marker, or may use the orientation marker together with, for example the control mark, to scan the image of the result region of the lateral flow test in a direction relative to the orientation marker to identify the test mark. The orientation marker may be a QR code, and therefore enable, for example, the hormone test to be linked to the user and/or the type of test.

The monitor may be any device capable of carrying out the required steps. For example the monitor may be a smartphone, tablet, laptop or other computing device. The monitor may comprise at least one processor configured to carry out instructions to cause the monitor to carry out some or all of the method or algorithm steps carried out by the monitor. The instructions to be carried out by the processor may be stored in the memory of the monitor. The instructions may be downloaded to the memory from a remote computer, for example in the form of an App for a smartphone.

The invention further provides a method of determining a hormone profile of a user, the method comprising:
i) using a monitor to notify the user that a hormone test of a hormone test set should be used within a window of the user's current menstrual cycle based on input received from the user;
ii) selecting the hormone test in accordance with the notification within the window and providing a sample to the hormone test to produce a test result indicative of the concentration of a hormone in the sample;
iii) capturing an image of the test result using the monitor;
iv) using the monitor to analyse the image of the test result to determine, quantitatively, the concentration of the hormone in the sample and store the determined concentration in a memory;
v) providing an output to the user which is indicative of the determined concentration.

The method may comprise repeating steps (i) to (v) for a plurality of hormone tests, each within a different window.

The method may comprise repeating steps (i) to (v) over a plurality of the user's menstrual cycles, for example at least three of the user's menstrual cycles, as this allows a more accurate hormone profile to be established.

### Examples of Suitable Hormone Tests

A progesterone assay may use a known type of lateral flow competitive format to detect pregnanediol glucuronide (PDG), the major urine metabolite of progesterone, in urine samples. The assay may utilise an anti-PDG antibody covalently conjugated to a carboxylated gold nanoshell particles, for example a 150 nm gold nanoshell particles, with antibody on the particle, for example with 10-40 μg/mL of antibody on the particle. The antibody-particle is then dried onto a conjugate pad. Upon sample addition, the conjugate is rehydrated and flows through a strip to interact with PDG, which is dried onto the test line area, for example at a concentration of 0.1-1 mg/mL. The PDG at the test line area may be bound to a carrier protein (for example Bovine Serum Albumin). PDG in the urine sample binds to the anti-PDG antibody-particle conjugate, competing for binding at the test line and inhibiting the signal. Thus, the test line signal intensity is inversely proportional to the amount of PDG in the sample. The anti-PDG antibody-particle conjugate flows past the test line area and interacts with a control line that contains an anti-Mouse IgG antibody. The anti-Mouse IgG antibody binds the PDG antibody-particle conjugate regardless of the presence of PDG to indicate proper test function.

An estrogen assay may use a known type of lateral flow competitive format to detect estrone-3-glucuronide (E3G), the major metabolite of estrogen, in urine samples. The assay may utilise an anti-E3G antibody covalently conjugated to a carboxylated gold nanoshell particles, for example a 150 nm gold nanoshell particles, with antibody on the particle, for example with 10-40 μg/mL of antibody on the particle. The antibody-particle conjugate is then dried onto the conjugate pad. Upon sample addition, the conjugate is rehydrated and flows through the strip to interact with E3G, which is dried onto the test line area, for example at a concentration of 0.1-1 mg/mL. The E3G at the test line area is bound to a carrier protein (for example Bovine Serum Albumin). E3G in the urine sample binds to the anti-E3G antibody-particle conjugate, competing for binding at the test line and inhibiting the signal. Thus, the test line signal intensity is inversely proportional to the amount of E3G in the sample. The anti-E3G antibody-particle conjugate flows past the test line area and interacts with a control line that contains an anti-Mouse IgG antibody. The anti-Mouse IgG antibody binds the E3G antibody-particle conjugate regardless of the presence of E3G to indicate proper test function.

A Follicle Stimulating Hormone assay may use a known type of lateral flow sandwich format to detect Follicle Stimulating Hormone (FSH) in urine samples. The assay may utilise an anti-FSH antibody specific to the beta portion of the FSH protein that is covalently conjugated to a carboxylated gold nanoshell particles, for example a 150 nm gold nanoshell particles, with antibody on the particle, for example with 10-40 μg/mL of antibody on the particle. The antibody-particle conjugate is then dried onto the conjugate pad. Upon sample addition, the conjugate is rehydrated and flows through the strip to interact with an anti-FSH antibody specific to the alpha portion of the FSH protein that is dried onto the test line area. FSH in the urine sample will bind to both the anti-FSH antibody-particle conjugate and the anti-FSH antibody at the test line. Thus, the test line signal intensity is proportional to the amount of FSH in the sample. The anti-FSH antibody-particle conjugate flows past the test line area and interacts with a control line that contains an anti-mouse IgG antibody. The anti-Mouse IgG antibody binds the FSH antibody-particle conjugate regardless of the presence of FSH to indicate proper test function.

The invention will now be described by way of example only with reference to the following figures in which:

FIG. 1 shows an image of a system for measuring a hormone profile;

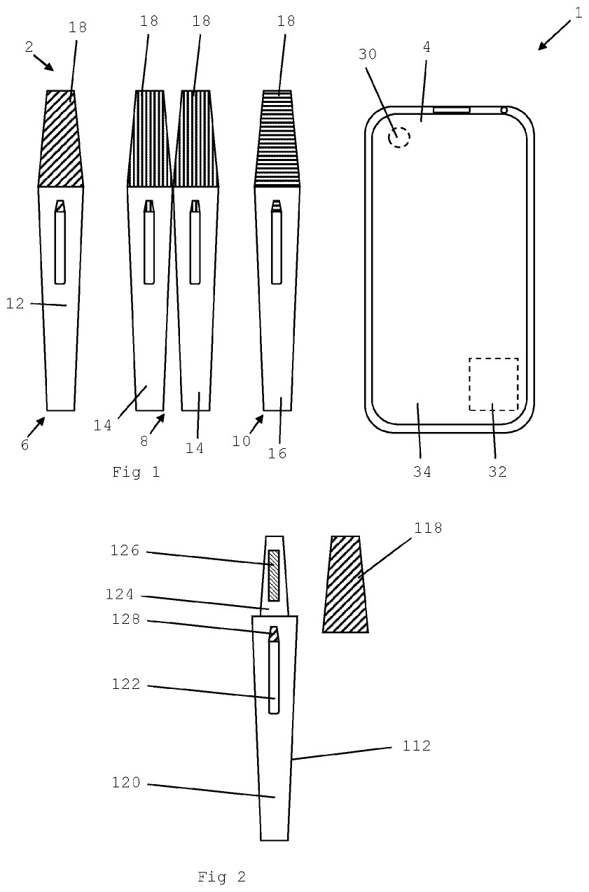

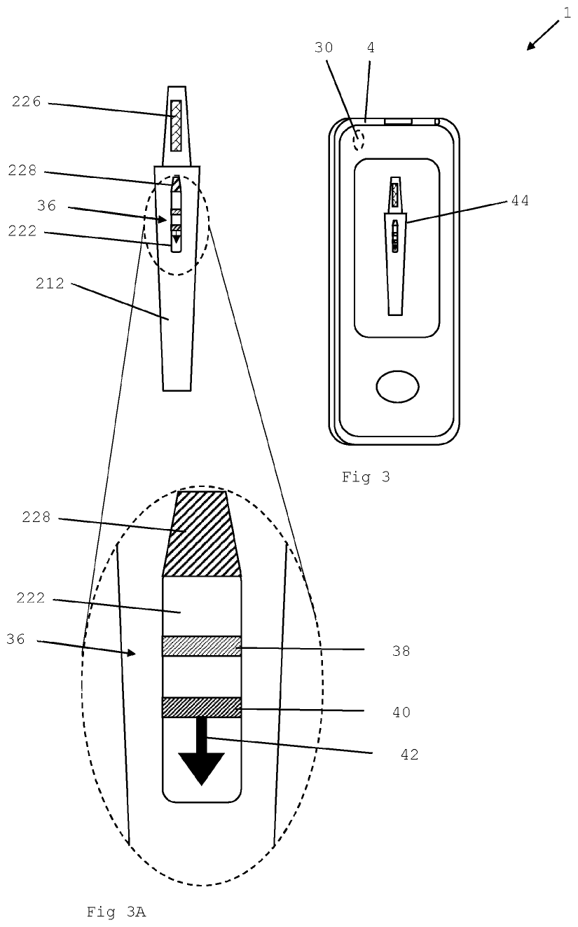

FIG. 2 shows an image of a hormone test for use in the system;

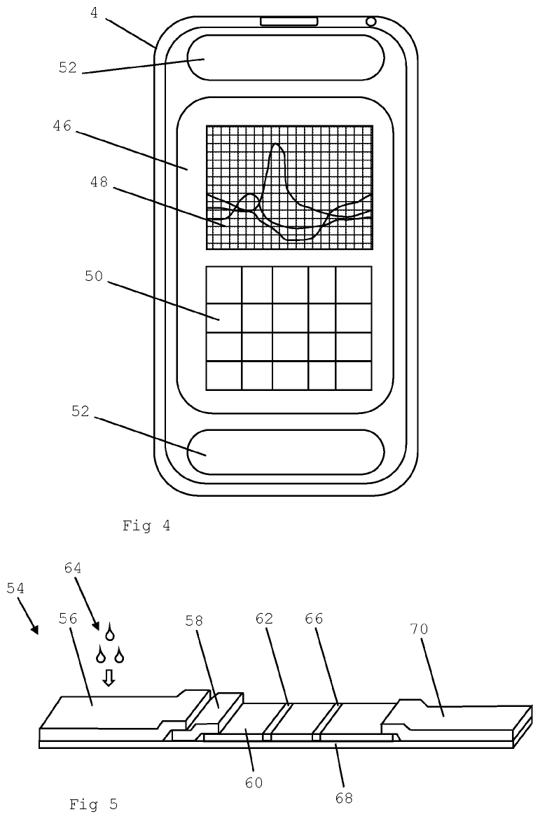

FIG. 3 shows an image of the system of FIG. 1 in use, with FIG. 3A showing an enlargement of a portion of a hormone test;

FIG. 4 shows an image of an output on a monitor;

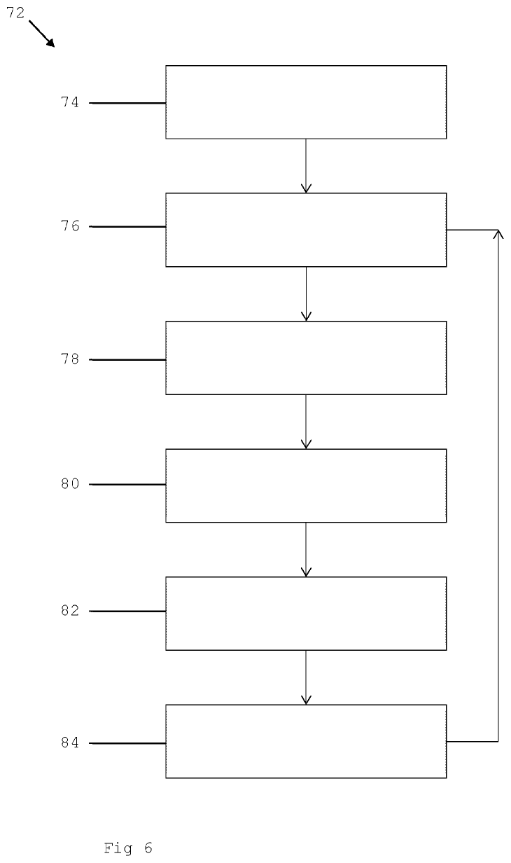

FIG. 5 shows schematic image of internal components of a hormone test;

FIG. 6 shows a flowchart of an example method of testing a hormone profile; and

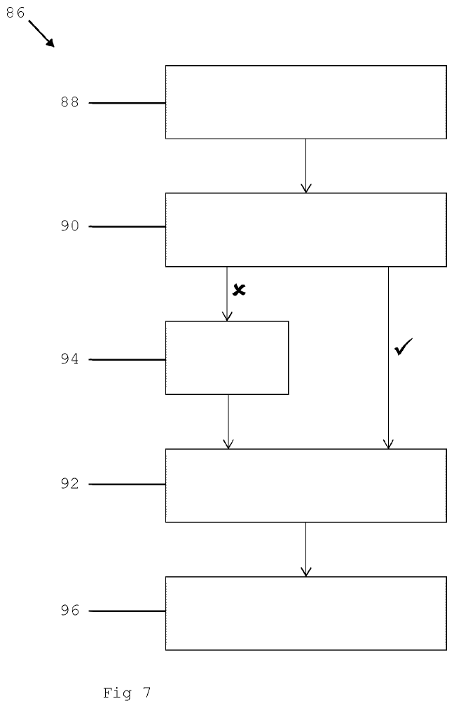

FIG. 7 shows a flowchart of an example of analysing an image in the hormone testing method of FIG. 6.

FIG. 1 shows an image of a system 1 for measuring a hormone profile of a user. The system 1 comprises a hormone test set 2 and a monitor 4.

The hormone test set 2 of this example comprises a first hormone test set 6, a second hormone test set 8 and a third hormone test set 10. The first hormone test set 6 comprises at least one first hormone test 12 for testing a first hormone, in this example follicle-stimulating hormone (FSH). The second hormone test set 8 comprises at least one second hormone test 14 for testing a second hormone, in this example is estrone-3-glucuronide (E3G). The third hormone test set 10 comprises at least one third hormone test 16 for testing a third hormone, in this example pregnanediol glucuronide (PDG). For monitoring a single menstrual cycle of a user the first hormone test set 6 may comprise a single first hormone test 12, the second hormone test set 8 may comprise two second hormone tests 14, and the third hormone test may comprise a single third hormone test 16.

It should be understood that the hormone test set 2 may comprise additional hormone test sets for testing other hormones, or each hormone test set may comprise additional hormone tests for testing on different days.

To differentiate between the first hormone test 12, second hormone tests 14, and third hormone tests 16 an indication may be provided on the hormone tests 12,14,16. In this example a cap 18 of the tests 12,14,16 has a colour dependent upon the type of hormone test. In other examples the indication may be a mark, word, symbol, texture, QR code or other indicia that can be detected by a user and/or the monitor. Alternatively, the first hormone test 12, second hormone tests 14, and third hormone tests 16 may be contained within a packaging which is removable by the user and may be differentiated from one another by an indication on the packaging. An example of a hormone test will now be described in more detail with reference to FIG. 2.

FIG. 2 shows an example of a hormone test 112, in this case a first hormone test 12 of the system of FIG. 1. In this example the hormone test 112 is a lateral flow assay which is intended to be used to collect a mid-flow urine sample from a user. The hormone test 112 comprises a body 120 which can be grasped by a user and a cap 118 which can be removably coupled to the body 120. The body 120 comprises a result window 122 and a test portion 124 which includes a sample receiving region 126. In this example the cap 118 covers the test portion 124 when coupled to the body 120, but in other examples it may cover, or restrict access to, portions of the test portion 124.

The body 120 of the hormone test 112 also includes an indicia 128 thereon which is indicative of the hormone test type. In this example the indicia 128 is a coloured mark which matches the colour of the cap 118. In another example, the indicia 128 may be a QR code. The indicia 128 may be visible in the image captured by the monitor 4 so that, when analysing the image, the monitor can determine that the correct test has been carried out. If no such indicia 128 is provided, or is not visible in the image, it may be assumed that the user has carried out the test that was scheduled, or the user may be asked to confirm which hormone test 6,8, 10,112 of the set of hormone tests 2 features in the image.

When the hormone test 112 is used, a test result is displayed in the result window 122. The test result is indicative of the concentration of the hormone in the sample. This will be explained in more detail in connection with later figures.

Referring back to FIG. 1, the monitor 4 is able to capture an image of the result of a test which, in this example, is displayed in the result window 122. In this example the monitor 4 is a smartphone which includes a camera 30 on a rear surface thereof. Based on the image of the test result the monitor is able to determine, quantitatively, the concentration of the hormone in the sample. The monitor then stores the determined concentration in a memory 32, in this case an internal, solid state memory. The monitor 4 can produce an output based on the determined concentration. In this example the monitor includes a display 34 and can produce a visible output thereon, but may produce an audible output, or an electronic output, for example to transmit the determined concentration to a different device.

The monitor 4 is further configured to receive an input from the user. The monitor may receive the input from a user in any suitable manner, for example via a typed input, audio input, visual input or electronic signal input that the user causes to be provided by another device. In this example the monitor 4 is a smartphone and the monitor is configured to receive the input via the touchscreen of the smartphone. Based on the input from the user the monitor is configured to determine and notify the user as to which of the plurality of hormone tests to use and capture an image of on which day of the user's current menstrual cycle. In this case the memory 32 includes a database of the minimum number of days on which users with a regular menstrual cycle should test which hormones in order to generate a hormone profile.

FIG. 3 shows an image of the system 1 of FIG. 1 in use. A hormone test 212 has had its cap removed and a sample of mid-flow urine has been collected on the sample receiving region 226.

Following sample collection a test result 36 has been displayed in the result window 222. The test result is shown in more detail in the enlargement of a portion of the hormone test 212 shown in FIG. 3A.

The test result 36 comprises a test mark 38, in this example a test line, and a control mark 40, in this example a control line. A feature of the test mark 38, in this example the intensity (but the feature could be any suitable feature, for example a colour) is dependent upon the concentration in the sample of the hormone being tested. In this example the test result 36 also includes an orientation mark 42, in this example an arrow. It should be understood that the orientation mark 42 may form a part of the test result 36 and only appear when the test is used. The orientation mark 42 may be separate from, or integral with, one, or both of, the test mark 38 and the control mark 40. In other examples the orientation mark 42 may be permanently visible in, or adjacent to, the result window 222. In some examples the indicia 228 which is indicative of the hormone test type may provide an orientation mark 42. The position of the indicia 228 relative to the result window 222 may be used to differentiate the test mark 38 from the control mark 40. For example, if the control mark 40 is below the test mark 38 and the indicia 228 is positioned below the result window 222 on the hormone test 212, then if the hormone test 212 is oriented such that indicia 228 is positioned below the result window 222 in the image of the hormone test 212, it can be inferred that the control mark 40 is the lowermost mark. Similarly, if the control mark 40 is below the test mark 38 and the indicia 228 is positioned above the result window 222 on the hormone test 212, then if the hormone test 212 is oriented such that indicia 228 is positioned above the result window 222 in the image of the hormone test 212, it can be inferred that the control mark 40 is the uppermost mark. To assist in taking the image of the hormone test 212, a frame may appear on display 34, within which the hormone test 212 should be positioned so that the image captures at least the indicia 228, which may be comprise a QR code, and the result window 222.

With the test result 36 now visible in the result window 222 the camera 30 of the monitor 4 is used to capture an image 44 of the hormone test 212 in which the result window 222 is visible. The monitor 4 may guide the user to take the image with optimal settings applied to capture a high-quality image. Such guidance may include instructing the user to use a camera flash. As the intensity of the test mark 38 is dependent upon the concentration in the sample of the hormone being tested, the image 44 can be analysed by the monitor 4 to provide a quantitative result. This can be achieved in a variety of ways, for example by comparing the intensity of the test mark 38 with the intensity of the control mark 40, optionally after some pre-processing of the image has been carried out to standardise the images, for example by correcting the white balance of the image. This could be based, for example, on the colour of the background of the test result 34, or on a part of the body of the hormone test, or on a standardised mark on the hormone test. The image may be further processed by changing, for example inverting, one or more colour channels to facilitate analysis of the test mark 38.

In the case of a faint test mark 38, which may not be visible to the eye, the image 44 may require additional processing to identify the test mark 38. The control mark 40 may be identified during a seeking process in which a detailed analysis of successive different portions of the test window 222 are carried out to try to locate the test mark 38 for subsequent analysis.

The analysis for the seeking process may be carried out on portions of the test window 222 which sequentially move further from, or closer to, the control mark 40 in a direction determined based upon the orientation mark 42. The direction for the seeking process may be determined based on the orientation mark 42 alone, for example it may itself be a mark indicative of the direction in which the test mark 38 should be found, for example an arrow, triangle, or other suitable shape. The direction for the seeking process may be determined based on the relative positions of the orientation mark 42 and the control mark 40.

In some examples the likely position of the test mark 38 in the image is determined based on the size of the control mark and/or orientation mark and/or any other feature of the hormone test visible in the image 44 and a seeking process may be started around that predicted location.

Two regions of interest may be captured: an area known to contain the result window 222 and an area known to contain the indicia 228, for example, a QR code. The monitor may then use the algorithm to carry out the following steps:
check the orientation of the hormone test 212, for example, by determining whether the indicia is above or below the result window 222;
rough crop the image to include the two regions of interest;
fine crop the image to exclude the indicia to enable the result window 222 to be analysed without interference from the rest of the image;
optionally, further crop the image to ensure only the result window 222 remains (this may be achieved by extracting an inner portion of the fine cropped image, for example, the inner 60% of the image);
change the image from the RGB colour space to the HSV colour space and select only the V channel, thereby producing a grayscale image;
denoise the image;
rotate the image by 90 degrees clockwise such that, provided the control mark 40 is below the test mark 40 in the result window 222, the control mark 40 is located on the left of the test mark 38;
determine, quantitively, the intensity of the control mark 40 and the test mark 38;
average the pixel intensity across each column of the pixel grid;
find 20th percentile values of the pixel intensities across each column of the pixel grid to identify the control lines against the background;
calculate the minimum value in the control mark 40 and the test mark 38;
normalise the minimum values to account for low lighting and shadow gradients using, for example, a linear regression model trained on “white” values; and
output the normalised values, thereby quantifying the intensity of the test mark 38.

A predictive model can be trained by running this method using images of test marks produced from known concentrations of hormones. If it is not possible to obtain a quantitative result which has sufficient accuracy, a qualitative interpretation (for example, low, normal or high) or an estimated range may be provided to the user.

Carrying out a detailed analysis only once the test mark 38 and control mark 40 have been identified reduces the processing power of the monitor required to carry out the analysis of the image 4. Reducing the area of the image 44 that is analysed in order to locate the test mark 38 has a similar advantage.

FIG. 4 shows an image of an output 46 on a monitor 4. In this example the output 46 comprises a graph 48 and chart 50 showing the hormone profile of the user over a period of time. The output 46 also comprises button 52 which may allow the user to provide input.

FIG. 5 shows a schematic image of internal components 54 of a hormone test. These internal components 54 are for a lateral flow assay and may be suitable for use in the plurality of hormone tests 2 of the system 1 of FIG. 1. The internal components 54 will be described in connection with a lateral flow progesterone assay based on detecting pregnanediol glucuronide, the major urine metabolite of progesterone, in urine samples, but it will be understood that similar internal components 54 can be used for other assays that function in a similar way.

The internal components 54 comprise a sample pad 56 to receive a liquid sample. A conjugate pad 58 comprising a dried anti-PDG antibody covalently conjugated to a carboxylated 150 nm gold nanoshell particles with 10-40 g/mL of antibody on the particles is arranged in contact with the sample pad 56.

When a urine sample 64 is received on the sample pad 56 the liquid passes into the conjugate pad 58 where the conjugate is rehydrated. The liquid sample and rehydrated conjugate flow along a membrane 60, in this example a nitrocellulose membrane, to a test line 62. The test line includes PDG, which is dried onto the test line area, for example at a concentration of 0.1-1 mg/mL. The PDG in the test line area may be bound to a carrier protein (for example Bovine Serum Albumin). If present, PDG in the urine sample binds to the anti-PDG antibody-particle conjugate, competing for binding at the test line and inhibiting a colour change. Thus, the test line signal intensity is inversely proportional to the amount of PDG in the sample.

The anti-PDG antibody-particle conjugate flows past the test line 62 and interacts with a control line 66 that contains an anti-Mouse IgG antibody. The anti-Mouse IgG antibody binds the PDG antibody-particle conjugate regardless of the presence of PDG to provide a colour change and thereby indicate proper test function.

The internal components 54 described above are arranged on a backing member 68 with the sample pad 56 arranged at one end of the backing 68, in the example a plastic backing, and a waste collection wick 70 arranged at an opposing end of the backing 68 to collect excess fluid from the sample.

FIG. 6 shows a flowchart 72 of an example method of testing a hormone profile of a user. The method may use the system described above. In step 74 a monitor receives an input from a user and based on that input determines a schedule on which hormone tests should be conducted for the user. The monitor then notifies the user of a window of the user's current menstrual cycle within which a particular hormone should be tested. The specified window may be a specified single day, or a plurality of days, for example two days or three days. The window may be less than a week.

In step 76 the user selects a hormone test in accordance with the notification and providing a sample to the hormone test to produce a test result indicative of the concentration of a hormone in the sample. As described above, the hormone test may be a lateral flow assay which is adapted to collect a sample of mid-flow urine. Once the sample has been provided to the hormone test the hormone test produces a test result indicative of the concentration of a hormone in the sample. The test result may be produced by the hormone test in fewer than 30 minutes, or in fewer than 15 minutes.

In step 78 a user uses the monitor to capture an image of the test result. As set out above, the monitor may be a smartphone, tablet or similar device, and capturing the image may comprise activating a camera of said device and using said camera to capture an image of the test result.

In step 80 the monitor analyses the image of the test result to determine, quantitatively, the concentration of the hormone in the sample and store the determined concentration in a memory.

In step 82 the monitor provides an output to the user which is indicative of the determined concentration.

In step 84 the monitor notifies the user of the next window of the user's current menstrual cycle within which a particular hormone should be tested.

FIG. 7 shows a flowchart 86 of an example of analysing an image in the hormone testing method 72 of FIG. 6.

In step 88 the image of the test result is received. The image of the test result may include some, or all, of the hormone test.

In step 90 the image of the test result is processed to determine whether the position of both a test mark and a control mark can be identified in the image using a first detection process. The control mark and the test mark may both be lines which, once the test is used, have a predetermined colour. The monitor may use an algorithm to process the image looking for lines of that predetermined colour, for example looking for red lines may be achieved by detecting red regions in the image using range of red colour in an HSV, or other, colour image.

The algorithm may filter the detected red regions to discard red regions which are of an inappropriate shape for the test or control marks. This may be based on the size or shape of the detected region.

The algorithm may then pair the detected marks using the geometric location and orientation. This may ensure that both a control and test mark have been detected in the image using the first detection process. As noted above, the orientation may be determined based on an orientation feature in the image, or through an assumption that the control mark is always above the test mark in the image, for example this may be how the user is instructed to capture the image.

If both a control and test mark have been detected in step 90 the method may proceed to step 92. However, if a test mark cannot be detected in the image, it may be because it is too faint to be detected by the first detection process. In such a situation the method may proceed to an intermediate step 94 in which a second detection process is carried out.

In step 94 a second detection process is carried out. The second detection process may be more sensitive and/or complex than the first detection process and may therefore be able to detect a fainter test mark.

The second detection process in step 94 may comprise identifying the control mark and then, based on the detected, or assumed, orientation of the hormone test in the image gradually move a detection area away from the control mark towards where a test line should be located and compute the differentiation of the mean colour in each area. The location of the test mark can be identified by the position in which a local maximum in the differentiation is identified.

With the test mark and control marks identified, either in step 90, or step 94, the method proceeds to step 92 in which the intensity of the test mark is analysed, possibly relative to the intensity of the control line. The intensity of the control line may be indicative of the concentration of the hormone in the sample.

The method then moves on to step 96 in which the detected intensity of the test mark is analysed to determine the concentration of the hormone in the sample and provide a quantitative result.

1. A system for determining a hormone profile of a user, the system comprising:
a hormone test set; and
a monitor, wherein:
the hormone test set comprises a plurality of hormone tests and each of the plurality of hormone tests is configured to be used to receive a sample from a user, detect a hormone in the sample, and display a test result indicative of the concentration of the hormone in the sample;
the monitor is able to capture an image of the test result, determine, quantitatively, the concentration of the hormone based on the image of the test result, store the determined concentration in a memory, and produce an output based on the determined concentration; and
the monitor is further configured to receive an input from the user and, based on the input, determine and notify the user as to which of the plurality of hormone tests to provide a sample to and capture an image of on which day of the user's current menstrual cycle.

2. The system of claim 1, wherein the input comprises the first date of the user's current menstrual cycle.

3. The system of claim 2, wherein the input further comprises the first date of the user's previous menstrual cycle, and the monitor is configured to notify the user as to which of the plurality of hormone tests to provide a sample to and capture an image of on which day based on the number of days of the user's previous menstrual cycle.

4. The system of claim 1, wherein the plurality of hormone tests comprise a first hormone test set comprising at least one first hormone test adapted to test a level of a first hormone.

5. The system of claim 4, wherein the first hormone test set comprises a plurality of first hormone tests, and wherein the user is not notified to provide a sample to and capture an image of one of the plurality of first hormone tests on every day of the user's current menstrual cycle.

6. The system of claim 4, wherein the plurality of hormone tests further comprise a second hormone test set comprising at least one second hormone test adapted to test a level of a second hormone, wherein the first hormone and second hormone are different hormones.

7. The system of claim 6, wherein the second hormone test set comprises a plurality of second hormone tests, and wherein the user is not notified to provide a sample to and capture an image of one of the plurality of second hormone tests on every day of the user's current menstrual cycle.

8. The system of claim 6, wherein the plurality of hormone tests further comprise a third hormone test set comprising at least one third hormone test adapted to test a third hormone level, wherein the first hormone, second hormone and third hormone are different hormones.

9. The system of claim 8, wherein the first hormone is follicle-stimulating hormone (FSH), the second hormone is estrone-3-glucuronide (E3G), and the third hormone is progesterone (PDG).

10. The system of claim 9, wherein the monitor is configured to notify the user to use and capture an image of the result of a first hormone test on a day when the user's level of FSH is expected to start increasing, a second hormone test on days when the user's level of E3G is expected to peak, and a third hormone test on a day when the user's level of PDG is expected to rapidly increase, wherein the days are dependent on the total number of days of the user's previous menstrual cycle.

11. The system of claim 10, wherein the output comprises a ratio of PDG to E3G.

12. The system of claim 1, wherein each of the plurality of hormone tests is a lateral flow test, wherein each of the lateral flow tests, in response to detecting the hormone in the sample, display a control line and a test line, wherein the test result comprises the test line, and the colour intensity of the test line is indicative of the concentration of the hormone in the sample.

13. The system of claim 12, wherein the lateral flow test comprises an orientation marker, and the monitor identifies the test line by identifying the orientation marker and scanning the lateral flow test in a direction relative to the orientation marker.

14. The system of claim 1, wherein the monitor is a smartphone.

15. A method of determining a hormone profile of a user, the method comprising:
i) using a monitor to determine and notify the user that a hormone test of a hormone test set should be used within a window of the user's current menstrual cycle based on input received from the user;
ii) selecting the hormone test in accordance with the notification within the window and providing a sample to the hormone test to produce a test result indicative of the concentration of a hormone in the sample;
iii) capturing an image of the test result using the monitor;
iv) using the monitor to analyse the image of the test result to determine, quantitatively, the concentration of the hormone in the sample and store the determined concentration in a memory;
v) providing an output to the user which is indicative of the determined concentration.

16. The method of claim 15, wherein the window is one day.

17. The method of claim 15, the method comprising repeating steps (i) to (v) for a plurality of hormone tests, each within a different window.

18. The method of claim 15, the method comprising repeating steps (i) to (v) over a plurality of the user's menstrual cycles.

19. The method of claim 18, the method comprising repeating steps (i) to (v) over three of the user's menstrual cycles.
